(19) United States
(12) Patent Application Publication
(10) Pub. No.: US 2008/0125461 A1
(43) Pub. Date: May 29, 2008

(54) (R,r)-Formoterol in Combination with Other Pharmacological Agents

(75) Inventor: Timothy J. Barberich, Concord, MA (US)

Correspondence Address: JONES DAY, 222 EAST 41ST ST, NEW YORK, NY, 10017, US

(73) Assignee: SEPRACOR INC., Marlborough, MA (US)

(21) Appl. No.: 11/547,687

(22) PCT Filed: Apr. 5, 2005
(86) PCT No.: PCT/US05/11489

§ 371 Date: Oct. 30, 2007

Related U.S. Application Data
(60) Provisional application No. 60/559,015, filed on Apr. 5, 2004.
(60) Provisional application No. 60/565,837, filed on Apr. 28, 2004.

(51) Int. Cl. A61K 31/47; A61K 31/135; A61K 31/40; A61P 11/06

(52) U.S. Cl. 514/311; 514/653; 514/419

(57) ABSTRACT

This invention related to methods of treating, preventing and managing various pulmonary diseases or disorders using stereomerically pure (R,R)-formoterol in combination with other pharmacological agents such as leukotriene inhibitors and neurokinin receptor antagonists. Pharmaceutical compositions comprising (R,R)-formoterol and other pharmacological agents are also disclosed.

This application claims priority to U.S. provisional application nos. 60/559,015, filed Apr. 5, 2004, and 60/565,837, filed Apr. 28, 2004, both of which are incorporated herein in their entireties by reference.

### 1. FIELD OF THE INVENTION

This invention relates to the use of stereomerically pure (R,R) formoterol in combination with other pharmacological agents for treating, preventing and managing various pulmonary diseases and disorders.

### 2. BACKGROUND OF THE INVENTION

Formoterol is a P₂-agonist, which is chemically named 2-hydroxy-5-[1-hydroxy-2-[[2-(4-methoxyphenyl)-1-methylethyl] -amino] ethyl] formanilide, and which has the following structure:

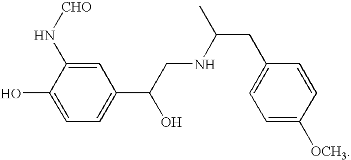

Formoterol has four stereoisomers, the mixture of which is commercially available under the trade name Foradil® (Novartis), which is indicated in the United States for helping prevent the symptoms of asthma. Unfortunately, the use of formoterol is associated with various side effects such as chills, cold- or flu-like symptoms, cough or hoarseness, fever, sneezing, sore throat, body aches or pain, chest pain, congestion, difficulty in breathing, headache, trauma, convulsions, decreased urine, and irregular heartbeat.

### 3. SUMMARY OF THE INVENTION

This invention encompasses methods of treating, preventing and managing pulmonary diseases or disorders comprising administering to a patient in need of such treatment, prevention or management a therapeutically or prophylactically effective amount of stereomerically pure (R,R)-formoterol, or a pharmaceutically acceptable salt, solvate, or prodrug thereof, and a therapeutically or prophylactically effective amount of a second pharmacological agent, or a pharmaceutically acceptable salt, solvate, or prodrug thereof.

This invention also encompasses pharmaceutical compositions comprising stereomerically pure (R,R)-formoterol, or a pharmaceutically acceptable salt, solvate, or prodrug thereof, and a second pharmacological agent, or a pharmaceutically acceptable salt, solvate, or prodrug thereof.

In one embodiment, the second pharmacological agent is a leukotriene inhibitor.

In one embodiment, the leukotriene inhibitor is a 5-lipoxygenase inhibitor.

In another embodiment, the leukotriene inhibitor is a 5-lipoxygenase activating protein antagonist.

In another embodiment, the leukotriene inhibitor is a leukotriene receptor antagonist.

In another embodiment, the second pharmacological agent is a neurokinin receptor antagonist.

In one embodiment, this invention also encompasses methods of treating, preventing and managing pulmonary diseases or disorders comprising administering to a patient in need of such treatment, prevention or management a therapeutically or prophylactically effective amount of stereomerically pure (R,R)-formoterol, or a pharmaceutically acceptable salt, solvate, or prodrug thereof, and a therapeutically or prophylactically effective amount of a second pharmacological agent, or a pharmaceutically acceptable salt, solvate, or prodrug thereof, while avoiding or reducing adverse effects associated with the administration of racemic or other stereoisomers of formoterol.

### 4. DETAILED DESCRIPTION OF THE INVENTION

This invention is based, in part, on a belief that stereomerically pure (R,R)-formoterol can be combined with other pharmacological agents, such as leukotriene inhibitors and neurokinin receptor antagonists, for the treatment, prevention, or management of pulmonary diseases and disorders. Without being limited by theory, this combination is believed to be more effective, have fewer adverse effects, and/or provide an overall improved therapeutic index as compared to prior methods of treating pulmonary diseases and disorders.

As used herein, the term “pharmaceutically acceptable salt” refers to salts prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lactic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like. Particularly preferred are hydrochloric, hydrobromic, phosphoric, and sulfuric acids, and most particularly preferred is the hydrochloride salt.

The term “solvate” means a compound of the present invention or a salt thereof, that further includes a stoichiometric or non-stoichiometric amount of solvent bound by non-covalent intermolecular forces. Where the solvent is water, the solvate is a hydrate.

As used herein, and unless otherwise specified, the term “prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO₂, —ONO, or —ONO₂ moieties.

As used herein, and unless otherwise specified, the terms “biohydrolyzable carbamate,” “biohydrolyzable carbonate,” “biohydrolyzable ureide” and “biohydrolyzable phosphate” mean a carbamate, carbonate, ureide and phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, aminoacids, hydroxyalkylamines, heterocyclic and heteroaromatic amines, and polyether amines.

### 4.1 Methods of Treatment, Prevention and Management

This invention encompasses methods of treating, preventing and managing pulmonary diseases or disorders comprising administering to a patient in need of such treatment, prevention or management a therapeutically or prophylactically effective amount of stereomerically pure (R,R)-formoterol, or a pharmaceutically acceptable salt, solvate, or prodrug thereof, and a therapeutically or prophylactically effective amount of a second pharmacological agent, or a pharmaceutically acceptable salt, solvate, or prodrug thereof.

This invention also encompasses methods of treating, preventing and managing pulmonary diseases or disorders comprising administering to a patient in need of such treatment, prevention or management a therapeutically or prophylactically effective amount of stereomerically pure (R,R)-formoterol, or a pharmaceutically acceptable salt, solvate, or prodrug thereof, and a therapeutically or prophylactically effective amount of a second pharmacological agent, or a pharmaceutically acceptable salt, solvate, or prodrug thereof, while avoiding or reducing adverse effects associated with the administration of racemic or other stereoisomers of formoterol. Examples of adverse effects include, but are not limited to, chills, cold- or flu-like symptoms, cough or hoarseness, fever, sneezing, sore throat, body aches or pain, chest pain, congestion, difficulty in breathing, headache, trauma, convulsions, decreased urine, and irregular heartbeat

As used herein, and unless otherwise indicated, the term “stereomerically pure” means a composition that comprises one stereoisomer of a compound and is substantially free of other stereoisomers of that compound. For example, a stereomerically pure composition of a compound having one stereocenter will be substantially free of the opposite stereoisomer of the compound. A stereomerically pure composition of a compound having two stereocenters will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and more preferably greater than about 97% by weight of one stereoisomer of the compound and less than about 3% by weight of the other stereoisomers, and even more preferably greater than about 99% by weight of one stereoisomer of the compound and less than about 1% by weight of the other stereoisomers of the compound.

The terms “treat,” “treating” and “treatment,” as used herein, contemplate an action that occurs while a patient is suffering from the specified disease or disorder, which reduces the severity of the disease or disorder, or retards or slows the progression of the disease or disorder.

As used herein, unless otherwise indicated, the terms “prevent,” “preventing” and “prevention” contemplate an action that occurs before a patient begins to suffer from the specified disease or disorder, which inhibits or reduces the severity of the disease or disorder. In this regard, the term “prevention” encompasses prophylactic administration of compounds or compositions of the invention.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein, and unless otherwise specified, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

In one embodiment, the second pharmacological agent is a leukotriene inhibitor.

Examples of leukotriene inhibitors that can be used in connection with methods of this invention include, but are not limited to, 5-lipoxygenase inhibitors, 5-lipoxygenase activating protein antagonists, and leukotriene receptor antagonists.

In one embodiment, leukotriene inhibitors used in methods and compositions of the invention are 5-lipoxygenase inhibitors. Examples of 5-lipoxygenase inhibitors include, but are not limited to, zileuton, docebenone, piripost and ICI-D2318.

In another embodiment, leukotriene inhibitors used in methods and compositions of the invention are 5-lipoxygenase activating protein antagonists. Examples of 5-lipoxygenase activating protein antagonists include, but are not limited to, MK-591 and MK-886.

In another embodiment, leukotriene inhibitors used in methods and compositions of the invention are leukotriene receptor antagonists. Examples of leukotriene receptor antagonists include, but are not limited to, zafirlukast, montelukast, pranlukast, sodium 1-(((R)-(3-(2-(6,7-difluoro-2-quinolinyl)ethynyl)phenyl-3-(2-(2-hydroxy-2-propyl)phenyl)thio)methyl)cyclopropaneacetate, 1-(((R)-(3-(2-(2,3-dichlorothieno[3,2-b]pyridin-5-yl)-(E)-ethenyl)phenyl)-3-(2-(1-hydroxy-1-methylethyl)phenyl)propyl)thio) methyl) cyclopropaneacetic acid, and (E)-8-[2-[4-[4-(4-fluorophenyl)butoxy]phenyl]ethenyl]-2-(1H-tetrazol-5-yl)-4H-1-benxopyran-4-one.

In one embodiment, the leukotriene receptor antagonist is montelukast. In a further embodiment, the leukotriene receptor antagonist is montelukast sodium.

In another embodiment, the leukotriene receptor antagonist is (E)-8-[2-[4-[4-(4-fluorophenyl)butoxy]phenyl]ethenyl]-2-(1H-tetrazol-5-yl)-4H-1-benxopyran-4-one.

In another embodiment, the second pharmacological agent is a neurokinin receptor antagonist. Examples of neurokinin receptor antagonists include, but are not limited to, cyclo[3-amino-L-alanyl-L-leucyl-N-[2-(acetylamino)-2-deoxy-p-D-glucopyranosyl-L-asparaginyl-L-alpha-aspartyl-L-tryptophyl-L-phenylalanyl]-(4-1)-lactam, Cam-2445, FK224, L-754,030, L-733,060, Ri 16301, SR48968, SR140333, SR142801, and ZD-6021. Chan et al., J. Pharm. Sci., 85(3): 251-7 (1996); Kraneveld et al., Am. J. Respir. Crit. Care Med., 156(2): 367-74 (1997); Navari et al., New Eng. J. Med., 340: 190-5 (1999); Grant et al., J. Physiol., 543: 1007-14 (2002); Mazzone et al., Am. J. Physiol. Regul. Integr. Comp. Physiol., 283(1): R86-R98 (2002); Yu et al., Annals of New York Academy of Sciences, 965: 247-53 (2002); Challet et al., Neuropharmacology, 40: 408-415; de Vries et al., Am. J. Respir. Crit. Care Med., 159: 1541-4 (1999); and Hamlin et al., Biology of Reproduction, 62: 1661-6 (2002). In a particular embodiment, the neurokinin receptor antagonist is cyclo[3-amino-L-alanyl-L-leucyl-N- [2-(acetylamino)-2-deoxy-β-D-glucopyranosyl--asparaginyl-L-alpha-aspartyl-L-tryptophyl-L-phenylalanyl]-(4-1 )-lactam.

Various pulmonary diseases or disorders can be treated, prevented and/or managed using methods of the invention. Examples of pulmonary diseases or disorders include, but are not limited to: respiratory failure; adult respiratory distress syndrome; chronic obstructive airway disorders such as, but not limited to, asthma, chronic obstructive pulmonary disease and giant bullae; acute bronchitis; chronic bronchitis; emphysema; reversible obstructive airway disease; nocturnal asthma; exercise induced bronchospasm; long-term maintenance treatment of asthma; prevention of bronchospasm in patients with reversible obstructive airway disease, including patients with symptoms of asthma, who require treatment with other inhaled short-acting β₂-antagonists; long-term management of bronchoconstriction associated with chronic obstructive pulmonary disease, including chronic bronchitis and emphysema; acute prevent of exercise-induced bronchospasm, used in occasional, as needed, basis; bronchiectasis; atelectasis; pulmonary embolism; pneumonia; lung abscess; hypersensitivity of the lung such as, but not limited to, hypersensitivity pneumonitis, eosinophilic pneumonias and allergic bronchopulmonary aspergillosis; and Goodpasture's syndrome.

Stereomerically pure (R,R)-formoterol, or a pharmaceutically acceptable salt, solvate, or prodrug thereof, and a second pharmacological agent, or a pharmaceutically acceptable salt, solvate, hydrate, clathrate or prodrug thereof, can be administered sequentially or concurrently.

In one embodiment, the stereomerically pure (R,R)-formoterol comprises at least about 80 percent, 90 percent, 95 percent, 97 percent, or 99 percent by weight of the total formoterol used. Stereomerically pure (R,R)-formoterol is preferably administered in an amount of from about 0.001 mg to about 50 mg per day, from about 0.002 mg to about 10 mg per day, or from about 0.003 mg to about 1 mg per day.

Suitable daily dosage ranges of the second pharmacological agents can be readily determined by those skilled in the art. See, e.g., Physician's Desk Reference (2001). For example, 5-lipoxygenase inhibitors can be administered at a daily dose range of from about 20 mg to about 2,500 mg per day, or from about 20 mg to about 800 mg per day. For leukotriene receptor antagonists, the daily dose can range from about 0.001 mg to about 100 mg, from about 0.002 mg to about 50 mg, from about 0.005 mg to about 10 mg, from about 0.01 mg to about 10 mg, from about 0.1 mg to about 5 mg, or from about 0.05 mg to about 1 mg per day. The particular amount of a leukotriene inhibitor will depend on the particular drug, as those of skill in the art are well aware. Similarly, suitable daily dosage ranges of neurokinin receptor antagonists can be readily determined by those skilled in the art. Typically, a neurokinin receptor antagonist may be administered in an amount from about 0.001 mg to about 1000 mg, from about 0.005 mg to about 500 mg, from about 0.01 mg to about 300 mg, from about 0.1 mg to about 200 mg, from about 0.1 mg to about 100 mg, from about 0.1 mg to about 50 mg, from about 1 mg to about 100 mg, from about 5 mg to about 50 mg, from about 1 mg to about 10 mg, from about 1 mg to about 20 mg, from about 5 mg to about 20 mg, or from about 0.1 mg to about 5 mg per day.

The selected dosage level and frequency of administration of the pharmaceutical compositions of the invention will depend upon a variety of factors including the route of administration, the time of administration, the rate of excretion of the therapeutic agents, the duration of the treatment, other drugs, compounds and/or materials used in the patient, the age, sex, weight, condition, general health and prior medical history of the patient being treated, and like factors well known in the medical arts. For example, the dosage regimen is likely to vary with pregnant women, nursing mothers and children relative to healthy adults. A physician having ordinary skill in the art can readily determine and prescribe the therapeutically effective amount of the pharmaceutical composition required.

Stereomerically pure (R,R)-formoterol can be synthesized using any suitable methods known in the art. For example, (R,R)-formoterol may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions, p. 268 (E. L. Eliel, Ed., Univ. of Notre Dame Press, Notre Dame, Ind., 1972), all of which are incorporated herein by reference.

4.2 Pharmaceutical Compositions

This invention encompasses pharmaceutical compositions comprising: stereomerically pure (R,R)-formoterol, or a pharmaceutically acceptable salt, solvate, or produrg thereof; a second pharmacological agent, or a pharmaceutically acceptable salt, solvate, or prodrug thereof; and a pharmaceutically acceptable carrier or excipient.

In one embodiment, the second pharmacological agent is a leukotriene inhibitor.

In one embodiment, the leukotriene inhibitor is a 5-lipoxygenase inhibitor.

In another embodiment, the leukotriene inhibitor is a 5-lipoxygenase activating protein antagonist.

In another embodiment, the leukotriene inhibitor is a leukotriene receptor antagonist.

In another embodiment, the second pharmacological agent is a neurokinin receptor antagonist.

Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic or hard gelatin capsules; cachets; troches; lozenges; dispersions; suppositories; ointments; cataplasms (poultices); pastes; powders; UDV nebulized solutions; dressings; creams; plasters; solutions; patches; aerosols ( e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

In one embodiment, the dosage form is a UDV nebulized solution. The solution may be water, and the solution may further comprise a stabilizer. See, e.g., U.S. Pat. No. 6,667,344, which is incorporated in its entirety by reference.

The formulation should suit the mode of administration. For example, oral administration may require enteric coatings to protect the compounds of this invention from degradation within the gastrointestinal tract. In another example, the compounds of this invention may be administered in a liposomal formulation to shield the compounds from degradative enzymes, facilitate transport in circulatory system, and effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington 's Pharmaceutical Sciences, 18th ed., Mack Publishing, Easton Pa. (1990).

The selected dosage level and frequency of administration of the pharmaceutical compositions of the invention will depend upon a variety of factors including the route of administration, the time of administration, the rate of excretion of the therapeutic agents, the duration of the treatment, other drugs, compounds and/or materials used in the patient, the age, sex, weight, condition, general health and prior medical history of the patient being treated, and like factors well known in the medical arts. For example, the dosage regimen is likely to vary with pregnant women, nursing mothers and children relative to healthy adults. A physician having ordinary skill in the art can readily determine and prescribe the therapeutically effective amount of the pharmaceutical composition required.

The pharmaceutical compositions of the invention may further comprise a pharmaceutically acceptable carrier. The term “pharmaceutically acceptable carrier” means one or more pharmaceutically acceptable excipients. Examples of such excipients are well known in the art and are listed in the USP (XXI)/NF (XVI), incorporated herein in its entirety by reference thereto, and include without limitation, binders, diluents, fillers, disintegrants, super disintegrants, lubricants, surfactants, antiadherents, stabilizers, and the like. The term “additives” is synonymous with the term “excipients” as used herein.

The term “pharmaceutically acceptable” is used herein to refer to those compounds, materials, compositions and/or dosage forms which are, within the scope of sound medical judgment, suitable for administration to and for use in contact with the tissues and fluids of human beings and animals without excessive toxicity, irritation, allergic response, or other problem or complication, commensurate with a reasonable medically sound benefit/risk ratio.

Further, the term “pharmaceutically acceptable” excipient is employed to mean that there are no untoward chemical or physical incompatibilities between the active ingredients and any of the excipient components of a given dosage form. For example, an untoward chemical reaction is one wherein the potency of (R,R)-formoterol or leukotriene inhibitor is detrimentally reduced or increased due to the addition of one or more excipients. Another example of an untoward chemical reaction is one wherein the taste of the dosage form becomes excessively sweet, sour or the like to the extent that the dosage form becomes unpalatable. Each excipient must be “acceptable” in the sense of being compatible with the other ingredients of the formulation and not injurious to the patient.

Physical incompatibility refers to incompatibility among the various components of the dosage form and any excipient(s) thereof. For example, the combination of the excipient(s) and the active ingredient(s) may form an excessively hygroscopic mixture or an excessively segregated mixture to the degree that the desired shape of the dosage form (e.g., tablet, troche etc.), its stability or the like cannot be sufficiently maintained to be able to administer the dosage form in compliance with a prescribed dosage regimen as desired.

It is noted that all excipients used in the pharmaceutical compositions or dosage forms made in accordance with the present invention preferably meet or exceed the standards for pharmaceutical ingredients and combinations thereof in the USP/NF. The purpose of the USP/NF is to provide authoritative standards and specifications for materials and substances and their preparations that are used in the practice of the healing arts. The USP/NF establish titles, definitions, descriptions, and standards for identity, quality, strength, purity, packaging and labeling, and also, where practicable, provide bioavailability, stability, procedures for proper handling and storage and methods for their examination and formulas for their manufacture or preparation.

The stability of a pharmaceutical product may be defined as the capability of a particular formulation, in a specific container, to remain within its physical, chemical, microbiological, therapeutic and toxicological specification, although there are exceptions, and to maintain at least about 90% of labeled potency level. Thus, for example, expiration dating is defined as the time in which the pharmaceutical product will remain stable when stored under recommended conditions.

Many factors affect the stability of a pharmaceutical product, including the stability of the therapeutic ingredient(s), the potential interaction between therapeutic and inactive ingredients and the like. Physical factors such as heat, light and moisture may initiate or accelerate chemical reactions.

4.2.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

Large-scale production of pharmaceutical compositions or dosage forms in accordance with the present invention may require, in addition to the therapeutic drug ingredients, excipients or additives including, but not limited to, diluents, binders, lubricants, disintegrants, colorants, flavors, sweetening agents and the like or mixtures thereof. By the incorporation of these and other additives, a variety of dosage forms (e.g., tablets, capsules, caplets, troches and the like) may be made. These include, for example, hard gelatin capsules, caplets, sugar-coated tablets, enteric-coated tablets to delay action, multiple compressed tablets, prolonged-action tablets, tablets for solution, effervescent tablets, buccal and sublingual tablets, troches and the like.

Hence, unit dose forms or dosage formulations of a pharmaceutical composition of the present invention, such as a troche, a tablet or a capsule, may be formed by combining a desired amount of each of the active ingredients with one or more pharmaceutically compatible or acceptable excipients, as described below, in pharmaceutically compatible amounts to yield a unit dose dosage formulation the desired amount of each active ingredient. The dose form or dosage formulation may be formed by methods well known in the art.

Tablets are often a preferred dosage form because of the advantages afforded both to the patient (e.g., accuracy of dosage, compactness, portability, blandness of taste as well as ease of administration) and to the manufacturer (e.g., simplicity and economy of preparation, stability as well as convenience in packaging, shipping and dispensing). Tablets are solid pharmaceutical dosage forms containing therapeutic drug substances with or without suitable additives.

Tablets are typically made by molding, by compression or by generally accepted tablet forming methods. Accordingly, compressed tablets are usually prepared by large-scale production methods while molded tablets often involve small-scale operations. For example, there are three general methods of tablet preparation: (1) the wet-granulation method; (2) the dry-granulation method; and (3) direct compression. These methods are well known to those skilled in the art. See, Remington's Pharmaceutical Sciences, 16th and 18th Eds., Mack Publishing Co., Easton, Pa. (1980 and 1990). See, also, U.S. Pharmacopeia XXI U.S. Pharmacopeial Convention, Inc., Rockville, Md. (1985).

Various tablet formulations may be made in accordance with the present invention. These include tablet dosage forms such as sugar-coated tablets, film-coated tablets, enteric-coated tablets, multiple-compressed tablets, prolonged action tablets and the like. Sugar-coated tablets (SCT) are compressed tablets containing a sugar coating. Such coatings may be colored and are beneficial in covering up drug substances possessing objectionable tastes or odors and in protecting materials sensitive to oxidation. Film-coated tablets (FCT) are compressed tablets that are covered with a thin layer or film of a water-soluble material. A number of polymeric substances with film-forming properties may be used. The film coating imparts the same general characteristics as sugar coating with the added advantage of a greatly reduced time period required for the coating operation. Enteric-coated tablets are also suitable for use in the present invention. Enteric-coated tablets (ECT) are compressed tablets coated with substances that resist dissolution in gastric fluid but disintegrate in the intestine. Enteric coating can be used for tablets containing drug substances that are inactivated or destroyed in the stomach, for those which irritate the mucosa or as a means of delayed release of the medication.

Multiple compressed tablets (MCT) are compressed tablets made by more than one compression cycle, such as layered tablets or press-coated tablets. Layered tablets are prepared by compressing additional tablet granulation on a previously compressed granulation. The operation may be repeated to produce multilayered tablets of two, three or more layers. Typically, special tablet presses are required to make layered tablets. See, for example, U.S. Pat. No. 5,213,738, incorporated herein in its entirety by reference thereto.

Press coated tablets are another form of multiple compressed tablets. Such tablets, also referred to as dry-coated tablets, are prepared by feeding previously compressed tablets into a tableting machine and compressing another granulation layer around the preformed tablets. These tablets have all the advantages of compressed tablets, i.e., slotting, monogramming, speed of disintegration, etc., while retaining the attributes of sugar coated tablets in masking the taste of the drug substance in the core tablet. Press-coated tablets can also be used to separate incompatible drug substances. Further, they can be used to provide an enteric coating to the core tablets. Both types of tablets (i.e., layered tablets and press- coated tablets) may be used, for example, in the design of prolonged-action dosage forms of the present invention.

Pharmaceutical compositions or unit dosage forms of the present invention in the form of prolonged-action tablets may comprise compressed tablets formulated to release the drug substance in a manner to provide medication over a period of time. There are a number of tablet types that include delayed-action tablets in which the release of the drug substance is prevented for an interval of time after administration or until certain physiological conditions exist. Repeat action tablets may be formed that periodically release a complete dose of the drug substance to the gastrointestinal fluids. Also, extended release tablets that continuously release increments of the contained drug substance to the gastrointestinal fluids may be formed.

In order for medicinal substances or therapeutic ingredients of the present invention, with or without excipients, to be made into solid dosage forms (e.g., tablets) with pressure, using available equipment, it is necessary that the material, either in crystalline or powdered form, possess a number of physical characteristics. These characteristics can include, for example, the ability to flow freely, as a powder to cohere upon compaction, and to be easily released from tooling. Since most materials have none or only some of these properties, methods of tablet formulation and preparation have been developed to impart these desirable characteristics to the material which is to be compressed into a tablet or similar dosage form.

As noted, in addition to the drugs or therapeutic ingredients, tablets and similar dosage forms may contain a number of materials referred to as excipients or additives. These additives are classified according to the role they play in the formulation of the dosage form such as a tablet, a caplet, a capsule, a troche or the like. One group of additives include, but are not limited to, binders, diluents (fillers), disintegrants, lubricants, and surfactants. In one embodiment the diluent, binder, disintegrant, and lubricant are not the same.

A binder is used to provide a free-flowing powder from the mix of tablet ingredients so that the material will flow when used on a tablet machine. The binder also provides a cohesiveness to the tablet. Too little binder will give flow problems and yield tablets that do not maintain their integrity, while too much can adversely affect the release (dissolution rate) of the drugs or active ingredients from the tablet. Thus, a sufficient amount of binder should be incorporated into the tablet to provide a free-flowing mix of the tablet ingredients without adversely affecting the dissolution rate of the drug ingredients from the tablet. With lower dose tablets, the need for good compressibility can be eliminated to a certain extent by the use of suitable diluting excipients called compression aids. The amount of binder used varies upon the type of formulation and mode of administration, and is readily discernible to those of ordinary skill in the art.

Binders suitable for use with dosage formulations made in accordance with the present invention include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (povidone), methyl cellulose, pre-gelatinized starch, hydroxypropyl methyl cellulose, (e.g., Nos. 2208, 2906, 2910), microcrystalline cellulose or mixtures thereof. Suitable forms of microcrystalline cellulose can include, for example, the materials sold as AVICEL-PH-101, AVICEL-PH-103 and AVICEL-PH-105 (available from FMC Corporation, American Viscose Division, Avicel Sales, Marcus Hook, Pa., U.S.A.).

Fillers or diluents are used to give the powder (e.g., in the tablet or capsule) bulk so that an acceptable size tablet, capsule or other desirable dosage form is produced. Typically, therapeutic ingredients are formed in a convenient dosage form of suitable size by the incorporation of a diluent therewith. As with the binder, binding of the drug(s) to the filler may occur and affect bioavailability. Consequently, a sufficient amount of filler should be used to achieve a desire d dilution ratio without detrimentally affecting release of the drug ingredients from the dosage form containing the filler. Further, a filler that is physically and chemically compatible with the therapeutic ingredient(s) of the dosage form should be used. The amount of filler used varies upon the type of formulation and mode of administration, and is readily discernible to those of ordinary skill in the art. Examples of fillers include, but are not limited to, lactose, glucose, sucrose, fructose, talc, calcium carbonate (e.g., granules or powder), microcrystalline cellulose, powdered cellulose, dextrates, kaolin, mannitol, silicic acid, sorbitol, starch, pre-gelatinized starch, or mixtures thereof.

Disintegrants are used to cause the dose form (e.g., tablet) to disintegrate when exposed to an aqueous environment. Too much of a disintegrant will produce tablets which may disintegrate in the bottle due to atmospheric moisture. Too little may be insufficient for disintegration to occur and may thus alter the rate and extent of release of drug(s) or active ingredient(s) from the dosage form. Thus, a sufficient amount of disintegrant that is neither too little nor too much to detrimentally alter the release of the drug ingredients should be used to form the dosage forms made according to the present invention. The amount of disintegrant used varies based upon the type of formulation and mode of administration, and is readily discernible to the skilled artisan. Examples of disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof.

When a dose form that dissolves fairly rapidly upon administration to the subject, e.g., in the subject's stomach is desired, a super disintegrant can be used, such as, but not limited to, croscarmellose sodium or sodium starch glycolate. The term “super disintegrant,” as used herein, means a disintegrant that results in rapid disintegration of drug or active ingredient in the stomach after oral administration. Use of a super disintegrant can facilitate the rapid absorption of drug or active ingredient(s) which may result in a more rapid onset of action.

Adhesion of the dosage form ingredients to the punches of the manufacturing machine (e.g., a tableting machine) must be avoided. For example, when drug accumulates on the punch surfaces, it causes the tablet surface to become pitted and therefore unacceptable. Also, sticking of drug or excipients in this way requires unnecessarily high ejection forces when removing the tablet from the die. Excessive ejection forces may lead to a high breakage rate and increase the cost of production not to mention excessive wear and tear on the dies. In practice, it is possible to reduce sticking by wet-massing or by the use of lubricants, e.g., magnesium stearate. However, selection of a drug salt with good anti-adhesion properties can also minimize these problems.

As noted, the lubricant is used to enhance the flow of the tableting powder mix to the tablet machine and to prevent sticking of the tablet in the die after the tablet is compressed. Too little lubricant will not permit satisfactory tablets to be made and too much may produce a tablet with a water-impervious hydrophobic coating, which can form because lubricants are usually hydrophobic materials such as stearic acid, magnesium stearate, calcium stearate and the like. Further, a water-impervious hydrophobic coating can inhibit disintegration of the tablet and dissolution of the drug ingredient(s). Thus, a sufficient amount of lubricant should be used that readily allows release of the compressed tablet from the die without forming a water-impervious hydrophobic coating that detrimentally interferes with the desired disintegration and/or dissolution of the drug ingredient(s).

Example of suitable lubricants for use with the present invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, or mixtures thereof. Additional lubricants include, for example, a syloid silica gel (AEROSIL 200, manufactured by W.R. Grace Co. of Baltimore Md.), a coagulated aerosol of synthetic silica (marketed by Deaussa Co. of Plano, Tex.), CAB-O-SIL (a pyrogenic silicon dioxide product sold by Cabot Co. of Boston, Mass.) or mixtures thereof.

Surfactants are used in dosage forms to improve the wetting characteristics and/or to enhance dissolution, and are particularly useful in pharmaceutical compositions or dosage forms containing poorly soluble or insoluble drug(s) or active ingredients. Examples of surfactants include, but are not limited to, polyoxyethylene sorbitan fatty acid esters, such as those commercially available as TWEENs (e.g. Tween 20) and Tween 80), polyethylene glycols, polyoxyethylene stearates, polyvinyl alcohol, polyvinylpyrrolidone, poly(oxyethylene)/ poly(oxypropylene) block co-polyers such as poloxamers (e.g., commercially available as PLURONICs), and tetrafunctional block copolymers derived from sequential addition of propylene oxide and ethylene oxide to ethylenediamine, such as polyxamines (e.g., commercially as TETRONICs (BASF)), dextran, lecithin, dialkylesters of sodium sulfosuccinic acid, such as Aerosol OT, sodium lauryl sulfate, alkyl aryl polyether sulfonates or alcohols, such as TRITON X-200 or tyloxapol, p-isononylphenoxypoly (glycidol) (e.g. Olin-10G or Surfactant 10-G (Olin Chemicals), or mixtures thereof. Other pharmaceutically acceptable surfactants are well known in the art, and are described in detail in the Handbook of Pharmaceutical Excipients.

Other classes of additives for use with the pharmaceutical compositions or dosage forms of the present invention include, but are not limited to, anti-caking or antiadherent agents, antimicrobial preservatives, coating agents, colorants, desiccants, flavors and perfumes, plasticizers, viscosity increasing agents, sweeteners, buffering agents, humectants and the like.

Examples of anti-caking agents include, but are not limited to, calcium silicate, magnesium silicate, silicon dioxide, colloidal silicon dioxide, talc, or mixtures thereof.

Examples of antimicrobial preservatives include, but are not limited to, benzalkonium chloride solution, benzethonium chloride, benzoic acid, benzyl alcohol, butyl paraben, cetylpyridinium chloride, chlorobutanol, cresol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymol, or mixtures thereof.

Examples of colorants for use with the present invention include, but are not limited to, pharmaceutically acceptable dyes and lakes, caramel, red ferric oxide, yellow ferric oxide or mixtures thereof. Examples of desiccants include, but are not limited to, calcium chloride, calcium sulfate, silica gel or mixtures thereof.

Flavors that may be used include, but are not limited to, acacia, tragacanth, almond oil, anethole, anise oil, benzaldehyde, caraway, caraway oil, cardamom oil, cardamom seed, compound cardamom tincture, cherry juice, cinnamon, cinnamon oil, clove oil, cocoa, coriander oil, eriodictyon, eriodictyon fluidextract, ethyl acetate, ethyl vanillin, eucalyptus oil, fennel oil, glycyrrhiza, pure glycyrrhiza extract, glycyrrhiza fluidextract, lavender oil, lemon oil, menthol, methyl salicylate, monosodium glutamate, nutmeg oil, orange flower oil, orange flower water, orange oil, sweet orange peel tincture, compound orange spirit, peppermint, peppermint oil, peppermint spirit, pine needle oil, rose oil, stronger rose water, spearmint, spearmint oil, thymol, tolu balsam tincture, vanilla, vanilla tincture, and vanillin or mixture thereof.

Examples of sweetening agents include, but are not limited to, aspartame, dextrates, mannitol, saccharin, saccharin calcium, saccharin sodium, sorbitol, sorbitol solution, or mixtures thereof.

Exemplary plasticizers for use with the present invention include, but are not limited to, castor oil, diacetylated monoglycerides, diethyl phthalate, glycerin, mono-and di-acetylated monoglycerides, polyethylene glycol, propylene glycol, and triacetin or mixtures thereof. Suitable viscosity increasing agents include, but are not limited to, acacia, agar, alamic acid, aluminum monostearate, bentonite, bentonite magma, carbomer 934, carboxymethylcellulose calcium, carboxymethylcellulose sodium, carboxymethylcellulose sodium 12, carrageenan, cellulose, microcrystalline cellulose, gelatin, guar gum, hydroxyethyl cellulose, hydroxypropyl cellulose, hydroxypropyl methylcellulose (Nos. 2208; 2906; 2910), magnesium aluminum silicate, methylcellulose, pectin, polyvinyl alcohol, povidone, silica gel, colloidal silicon dioxide, sodium alginate, tragacanth and xanthan gum or mixtures thereof.

Buffering agents that may be used in the present invention include, but are not limited to, magnesium hydroxide, aluminum hydroxide and the like, or mixtures thereof. Examples of humectants include, but are not limited to, glycerol, other humectants or mixtures thereof.

The dosage forms of the present invention may further include one or more of the following: (1) dissolution retarding agents, such as paraffin; (2) absorption accelerators, such as quaternary ammonium compounds; (3) wetting agents, such as, for example, cetyl alcohol and glycerol monostearate; (4) absorbents, such as kaolin and bentonite clay; (5) antioxidants, such as water soluble antioxidants (e.g., ascorbic acid, cysteine hydrochloride, sodium bisulfate, sodium metabisulfate, sodium sulfite and the like), oil soluble antioxidants (e.g., ascorbyl palmitate, hydroxyanisole (BHA), butylated hydroxy toluene (BHT), lecithin, propyl gallate, alpha-tocopherol and the like); and (6) metal chelating agents, such as citric acid, ethylenediamine tetracetic acid (EDTA), sorbitol, tartaric acid, phosphoric acid and the like.

Dosage forms of the present invention, such as a tablet or caplet, may optionally be coated. Inert coating agents typically comprise an inert film-forming agent dispersed in a suitable solvent, and may further comprise other pharmaceutically acceptable adjuvants, such as colorants and plasticizers. Suitable inert coating agents, and methods for coating, are well known in the art, including without limitation aqueous or non-aqueous film coating techniques or microencapsulation. Examples of film-forming or coating agents include, but are not limited to, gelatin, pharmaceutical glaze, shellac, sucrose, titanium dioxide, carnauba wax, microcrystalline wax, celluloses, such as methylcellulose, hydroxymethyl cellulose, carboxymethylcellulose, cellulose acetate phthalate, hydroxypropyl methylcellulose (e.g., Nos.: 2208, 2906, 2910), hydroxypropyl cellulose, hydroxypropyl methyl cellulose phthalate (e.g., Nos.: 200731, 220824), hydroxyethylcellulose, methylhydroxyethylcellulose, ethylcellulose which may optionally be cross-linked, and sodium carboxymethyl cellulose; vinyls, such as polyvinyl pyrrolidione, polyvinyl acetate phthalate,; glycols, such as polyethylene glycols; acrylics, such as dimethylaminoethyl methacrylate-methacrylate acid ester copolymer, and ethylacrylate-methylmethacrylate copolymer; and other carbohydrate polymers, such as maltodextrins, and polydextrose, or mixtures thereof. The amount of coating agent and the carrier vehicle (aqueous or non-aqueous) used varies upon the type of formulation and mode of administration, and is readily discernible to those of ordinary skill in the art.

A coating of a film forming polymer may optionally be applied to a tablet or caplet (e.g., a capsule shaped tablet) in accordance with the present invention by using one of several types of equipment such as a conventional coating pan, Accelacota, High-Cola or Worster air suspension column. Such equipment typically has an exhaust-system to remove dust and solvent or water vapors to facilitate quick drying. Spray guns or other suitable atomizing equipment may be introduced into the coating pans to provide spray patterns conducive to rapid and uniform coverage of the tablet bed. Normally, heated or cold drying air is introduced over the tablet bed in a continuous or alternate fashion with a spray cycle to expedite drying of the film coating solution.

The coating solution may be sprayed by using positive pneumatic displacement or peristaltic pump systems in a continuous or intermittent spray-dry cycle. The particular type of spray application is selected depending upon the drying efficiency of the coating pan. In most cases, the coating material is sprayed until the tablets are uniformly coated to the desired thickness and the desired appearance of the tablet is achieved. Many different types of coatings may be applied such as enteric, slow release coatings or rapidly dissolving type coatings for fast acting tablets. Preferably, rapidly dissolving type coatings are used to permit more rapid release of the active ingredients, resulting in hastened onset. The thickness of the coating of the film forming polymer applied to a tablet, for example, may vary. However, it is preferred that the thickness simulate the appearance, feel (tactile and mouth feel) and function of a gelatin capsule. Where more rapid or delayed release of the therapeutic agent(s) is desired, one skilled in the art would easily recognize the film type and thickness, if any, to use based on characteristics such as desired blood levels of active ingredient, rate of release, solubility of active ingredient, and desired performance of the dosage form.

A number of suitable film forming agents for use in coating a final dosage form, such as tablets include, for example, methylcellulose, hydroxypropyl methyl cellulose (PHARMACOAT 606 6 cps), polyvinylpyrrolidone (povidone), ethylcellulose (ETHOCEL 10 cps), various derivatives of methacrylic acids and methacrylic acid esters, cellulose acetate phthalate or mixtures thereof.

The method of preparation and the excipients or additives to be incorporated into dosage form (such as a tablet or caplet) are selected in order to give the tablet formulation the desirable physical characteristics while allowing for ease of manufacture (e.g., the rapid compression of tablets). After manufacture, the dose form preferably should have a number of additional attributes, for example, for tablets, such attributes include appearance, hardness, disintegration ability and uniformity, which are influenced both by the method of preparation and by the additives present in the tablet formulation.

Further, it is noted that tablets or other dosage forms of the pharmaceutical compositions of the invention should retain their original size, shape, weight and color under normal handling and storage conditions throughout their shelf life. Thus, for example, excessive powder or solid particles at the bottom of the container, cracks or chips on the face of a tablet, or appearance of crystals on the surface of tablets or on container walls are indicative of physical instability of uncoated tablets. Hence, the effect of mild, uniform and reproducible shaking and tumbling of tablets should be undertaken to insure that the tablets have sufficient physical stability. Tablet hardness can be determined by commercially available hardness testers. In addition, the in vitro availability of the active ingredients should not change appreciably with time.

The tablets, and other dosage forms of the pharmaceutical compositions of the present invention, such as dragees, capsules, pills and granules, may optionally be scored or prepared with coatings and shells, such as enteric coatings and other coatings well known in the pharmaceutical formulating art.

4.2.2 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients (i.e., the compounds of this invention) disclosed herein can also be incorporated into the parenteral dosage forms of the invention.

4.2.3 Transdermal, Topical and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied.

Depending on the specific tissue to be treated, additional components m ay be used prior to, in conjunction with, or subsequent to treatment with active ingredients of the invention. For example, penetration enhancers can be used to assist in delivering the active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

4.2.4 Compositions with Enhanced Stability

The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients.

4.2.5 Delayed Release Dosage Forms

Active ingredients of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profit e in varying proportions. Suitable controlled-release formulations known to those of ordinary skill in the art, including those described herein, can be readily selected for use with the compounds of this invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

4.2.6 Kits

In some cases, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a single unit dosage form of the compounds of this invention, or a pharmaceutically acceptable salt, hydrate, prodrug, solvate, or clathrate thereof, and a single unit dosage form of another agent that may be used in combination with the compounds of this invention. Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The invention is further defined by reference to the following non-limiting examples. It will be apparent to those skilled in the art that many modifications, both to materials and methods, can be practiced without departing from the spirit and scope of this invention.

### 5. EXAMPLES

The following examples illustrate specific pharmaceutical compositions of the invention.

### 5.1 Example 1

| | | |
|---|---|---|
| (R,R)-formoterol | 4.5 | μg |
| Zafirlukast | 100 | μg |
| Lactose monohydrate | 0.2-2 | mg |

### 5.2 Example 2

| | | |
|---|---|---|
| (R,R)-formoterol | 9.0 | μg |
| Zafirlukast | 100 | μg |
| Lactose monohydrate | 0.2-2 | mg |

### 5.3 Example 3

| | | |
|---|---|---|
| (R,R)-formoterol | 4.5 | μg |
| Zafirlukast | 200 | μg |
| Lactose monohydrate | 0.3-2 | mg |

### 5.4 Example 4

| | | |
|---|---|---|
| (R,R)-formoterol | 9.0 | μg |
| Zafirlukast | 200 | μg |
| Lactose monohydrate | 0.3-2 | mg |

### 5.5 Example 5

| | | |
|---|---|---|
| (R,R)-formoterol | 4.5 | μg |
| Montelukast sodium | 50 | μg |
| Lactose monohydrate | 0.2-2 | mg |

### 5.6 Example 6

| | | |
|---|---|---|
| (R,R)-formoterol | 4.5 | μg |
| Montelukast sodium | 100 | μg |
| Lactose monohydrate | 0.2-2 | mg |

All of the patents, patent applications and publications referred to in this application are incorporated herein in their entireties. Moreover, citation or identification of any reference in this application is not an admission that such reference is available as prior art to this invention. The full scope of the invention is better understood with reference to the appended claims.

1. A method of treating or managing a pulmonary disease or disorder which comprises administering to a patient a therapeutically effective amount of stereomerically pure (R,R) formoterol, or a pharmaceutically acceptable salt or solvate thereof, and a therapeutically effective amount of a leukotriene inhibitor, or a pharmaceutically acceptable salt or solvate thereof.

2-3. (canceled)

4. The method of claim 1, wherein stereomerically pure (R,R) formoterol, or a pharmaceutically acceptable salt or solvate thereof, and the leukotriene inhibitor, or a pharmaceutically acceptable salt or solvate thereof, are concurrently administered.

5. The method of claim 1, wherein stereomerically pure (R,R) formoterol, or a pharmaceutically acceptable salt or solvate thereof, and the leukotriene inhibitor, or a pharmaceutically acceptable salt or solvate thereof, are sequentially administered.

6. The method of claim 1, wherein the leukotriene inhibitor is a 5-lipoxygenase inhibitor, 5-lipoxygenase activating protein antagonist, or a leukotriene receptor antagonist.

7. The method of claim 6, wherein the leukotriene inhibitor is a 5-lpoxygenase inhibitor.

8. The method of claim 7, wherein the 5-lipoxygenase inhibitor is zileuton, docebenone, piripost or ICI-D2318.

9. The method of claim 6, wherein the leukotriene inhibitor is a 5-lipoxygenase activating protein antagonist.

10. The method of claim 9, wherein the 5-lipoxygenase activating protein antagonist is MK-591 or MK-886.

11. The method of claim 6, wherein the leukotriene inhibitor is a leukotriene receptor antagonist.

12. The method of claim 11, wherein the leukotriene receptor antagonist is zafirlukast, montelukast, pranlukast, sodium 1-(((R)-(3-(2-(6,7-difluoro-2-quinolinyl)ethynyl)phenyl-3-(2-(2-hydroxy-2-propyl)phenyl)thio)methyl) cyclopropaneacetate, 1-(((R)-(3-(2-(2,3-dichlorothieno[3,2-b]pyridin-5-yl)-(E)-ethenyl)phenyl)-3-(2-(1-hydroxy- 1-methylethyl)phenyl)propyl)thio)methyl) cyclopropaneacetic acid, or (E)-8-[2-[4-[4-(4-fluorophenyl)butoxy]phenyl]ethenyl]-2-(1H-tetrazol-5-yl)-4H- 1-benxopyran-4-one.

13. A method of treating or managing a pulmonary disease or disorder which comprises administering to a patient a therapeutically effective amount of stereomerically pure (R,R) formoterol, or a pharmaceutically acceptable salt or solvate thereof, and a therapeutically effective amount of a neurokinin receptor antagonist, or a pharmaceutically acceptable salt or solvate thereof.

14-15. (canceled)

16. The method of claim 13, wherein stereomerically pure (R,R) formoterol, or a pharmaceutically acceptable salt or solvate thereof, and the neurokinin receptor antagonist, or a pharmaceutically acceptable salt or solvate thereof, are concurrently administered.

17. The method of claim 13, wherein stereomerically pure (R,R) formoterol, or a pharmaceutically acceptable salt or solvate thereof, and the neurokinin receptor antagonist, or a pharmaceutically acceptable salt or solvate thereof, are sequentially administered.

18. The method of 13, wherein the neurokinin receptor antagonist is cyclo[3-amino-L-alanyl-L-leucyl-N-[2-(acetylamino)-2-deoxy-β-D-glucopyranosyl-L-asparaginyl-L-alpha-aspartyl-L-tryptophyl-L-phenylalanyl]-(4-1)-lactam, Cam-2445, FK224, L-754,030, L-733,060, R116301, SR48968, SR140333, SR142801, or ZD-6021.

19. The method of claim 18, wherein the neurokinin receptor antagonist is cyclo[3-amino-L-alanyl-L-leucyl-N-[2-(acetylamino)-2-deoxy-β-D-glucopyranosyl-L-asparaginyl-L-alpha-aspartyl-L-tryptophyl-L-phenylalanyl]-(4-1)-lactam.

20. The method of claim 1 or 13, wherein the pulmonary disease or disorder is respiratory failure; adult respiratory distress syndrome; chronic obstructive airway disorders such as, but not limited to, asthma, chronic obstructive pulmonary disease and giant bullae; acute bronchitis; chronic bronchitis; emphysema; reversible obstructive airway disease; nocturnal asthma; exercise induced bronchospasm; bronchiectasis; atelectasis; pulmonary embolism; pneumonia; lung abscess; hypersensitivity of the lung; or Goodpasture's syndrome.

21. The method of claim 1 or 13, wherein the treatment is long-term maintenance treatment of asthma.

22. (canceled)

23. The method of claim 1 or 13, wherein the management is long-term management of bronchoconstriction associated with chronic obstructive pulmonary disease.

24. The method of claim 23, wherein the chronic obstructive pulmonary disease is chronic bronchitis or emphysema.

25. The method of claim 20, wherein the hypersensitivity of the lung is hypersensitivity pneumonitis, eosinophilic pneumonias or allergic bronchopulmonary aspergillosis.

26. The method of claim 20, wherein the pulmonary disease or disorder is a chronic obstructive airway disorder.

27. The method of claim 26, wherein the chronic obstructive airway disorder is asthma or chronic obstructive pulmonary disease.

28-43. (canceled)
